(19) United States
(12) Patent Application Publication
(10) Pub. No.: US 2016/0206463 A1
(43) Pub. Date: Jul. 21, 2016

(54) Arch Support Brace

(71) Applicant: Jeffrey J. Watson, Bloomington, MN (US)

(72) Inventor: Jeffrey J. Watson, Bloomington, MN (US)

(21) Appl. No.: 14/996,507

(22) Filed: Jan. 15, 2016

Related U.S. Application Data
(60) Provisional application No. 62/104,165, filed on Jan. 16, 2015.

(51) Int. Cl. A61F 5/01; A61F 5/14

(52) U.S. Cl. CPC A61F 5/0111; A61F 5/14

(57) ABSTRACT

The device is comprised of a generic cushioned shock absorbing type material orthotic support structure attached to the brace layer. The orthotic support structure comprises a raised midsole that tapers at the anterior end and transitions into a heel cushion which surrounds the bottom of the wearer's heel at the posterior end. The orthotic support structure continues to the heel, where it provides a cup to form to the shape of the heel.

This non-provisional application claims the benefit of provisional application No. 62/104,165 filed on Jan. 16, 2015.

### BACKGROUND OF INVENTION

Plantar fasciitis is one of the most common foot injuries obtained in the United States, estimated to affect more than 1 million people per year. The injury is most often from chronic or long-term high levels of stress placed on the plantar fascia and or the flexor digitorum brevis muscle. These injuries can range from mild to severe, even resulting in surgery in severe cases. Pain is noted to be worst in the morning, within the first few steps of weight bearing. Due to the fact that the average human takes around 5000 steps per day, providing people with an option to maintain the ability to walk without pain is a key part of solving the issue.

Treatment for plantar fasciitis normally consists of rest, ice, compression, and elevation. These are conservative treatments, and generally have only fair results. To have a greater impact on the pain aspect of the foot, often orthotics are prescribed.

Most physicians will recommend an orthotic for patients suffering from plantar fasciitis. Orthotics range from over the counter, to generic, firm, soft, semi-rigid, and custom orthotics. Most provide at least minimal relief. Price ranges also vary, with custom generally occurring the most cost, with the cheapest being generic brands or over the counter.

This device differs very much from others in that it is a custom or generic orthotic built into a brace. The built in feature allows a patient to place the brace on and wear it barefoot, or with most types of shoes. This greatly increases ease for the patient, and also allows them to have an orthotic device on while they are barefoot, which may be the majority of the day.

Another goal of this device is to provide substantial support to improve gait mechanics. With this, not only will pain be relieved, but over time the function of the foot and mechanics of the gait cycle may improve as well.

This device aims to provide a short-term pain relief device along with a long-term solution to improve the gait cycle.

### DESCRIPTION OF INVENTION

This invention depicts a footwear device with multiple purposes, primarily relieving arch pain by providing a custom or generic orthotic support structure which is a semi-rigid or soft orthotic having shock absorbing qualities, attached to a brace layer, and a method to apply the device. The purpose of this device is to promote proper foot mechanics, hence improving gait and providing relief for tension on the plantar fascia. This device can be worn with or without shoes, and will be able to be worn with many types of shoes. The device requires skin contact and if socks are worn, they must be worn over the top (exterior) of the device. The device is lightweight, has no restriction on range of motion of the joints of the ankle and foot, and is not bulky, which allows the device to be versatile and worn during high activity levels.

The device is comprised of a generic cushioned shock absorbing type material orthotic support structure attached to the brace layer. The orthotic support structure comprises a raised midsole that tapers at the anterior end and transitions into a heel cushion which surrounds the bottom of the wearer's heel at the posterior end. The orthotic support structure continues to the heel, where it provides a cup to form to the shape of the heel. The orthotic support structure is continuous with the brace layer. There is no support for the toes or metatarsal-phalange joints. The device provides continuous support that is worn around the majority of the foot and up the ankle, ending just above the medial and lateral malleoli proximally and ending near the metatarsal heads distally.

The brace layer comprises a non-rigid, flexible material with elastic properties that acts as the component to which the orthotic support structure is attached to, and also is the main configuration to the wearer's foot. The material for the brace layer needs to be flexible and elastic in nature, but also breathable. Along with having the ability to be breathable, it may be washable as well. The bottom of the device has a gripping texture to prevent slipping of the foot on low friction flooring; this can be a separate layer, or just the orthotic support structure itself with a rubbery or gripping texture.

The device is designed to fit a range of foot sizes, and will have elastic or stretch properties that allow it to stay snug and compressed to the user's foot. The orthotic support structure can be completely surrounded by the brace layer, however in the preferred embodiment the orthotic support structure is attached to the bottom of the brace layer, thereby allowing for the orthotic brace layer material itself to act as the gripping or higher friction material to help prevent slippage on a low friction surface when contacting the ground during gait. The orthotic support structure itself is preferably comprised of a foam or shock absorbing material that can disperse the force of the foot during the gait cycle. This material is resistant to deformation with wear. The orthotic support structure continues to cover the heel, where it becomes thinner to prevent it from excessively changing the angle of the talocrural joint. The orthotic support structure is tapered at its anterior end, and does not reach the metatarsal heads. The sides and heel aspect of the orthotic support structure are tapered at their superior aspect, and become a continuation with the brace, which continues around the entire ankle. The orthotic support structure is sewn, attached, constructed with, or adhered to the brace so that it will not be moveable within the brace. The bottom of the brace is layered or composed of a material with an adhesive or gripping property to allow the brace to maintain traction on slippery or low friction surfaces. Alternatively, the brace layer can surround the orthotic support structure, which would allow the bottom of the brace layer to comprise the traction element.

### DRAWINGS

FIG. 1 depicts a frontal view of the arch support brace displayed on both of a wearer's feet.

FIG. 2 depicts a medial view of the brace shown in FIG. 1 as displayed on a wearer's right foot.

FIG. 3 depicts a bottom view of the orthotic support structure and brace depicted in FIG. 1 displayed on a wearer's right foot.

FIG. 4 depicts a lateral view of the orthotic support structure and brace displayed on a wearer's left foot.

FIG. 5 depicts a medial superior oblique view of the brace layer only.

FIG. 6 depicts a frontal view of the orthotic support structure only.

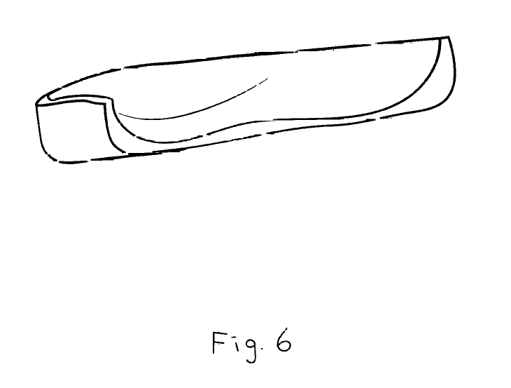

FIG. 7 depicts a medial superior oblique view of the orthotic support structure only.

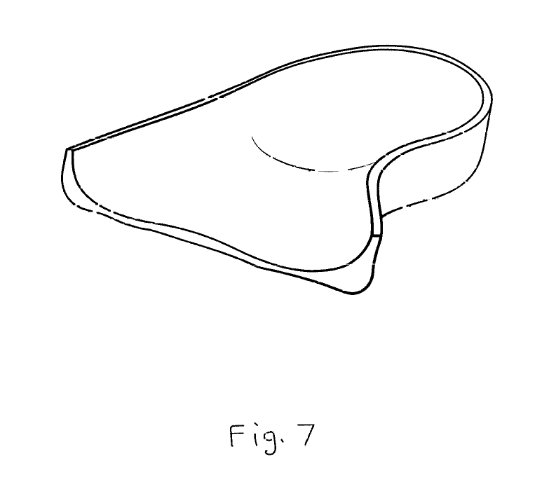

FIG. 8 depicts a medial superior oblique view of the orthotic support structure and brace layer.

FIG. 9 depicts a medial bottom oblique view of the orthotic support structure and brace layer.

### DETAILED DESCRIPTION OF DRAWINGS

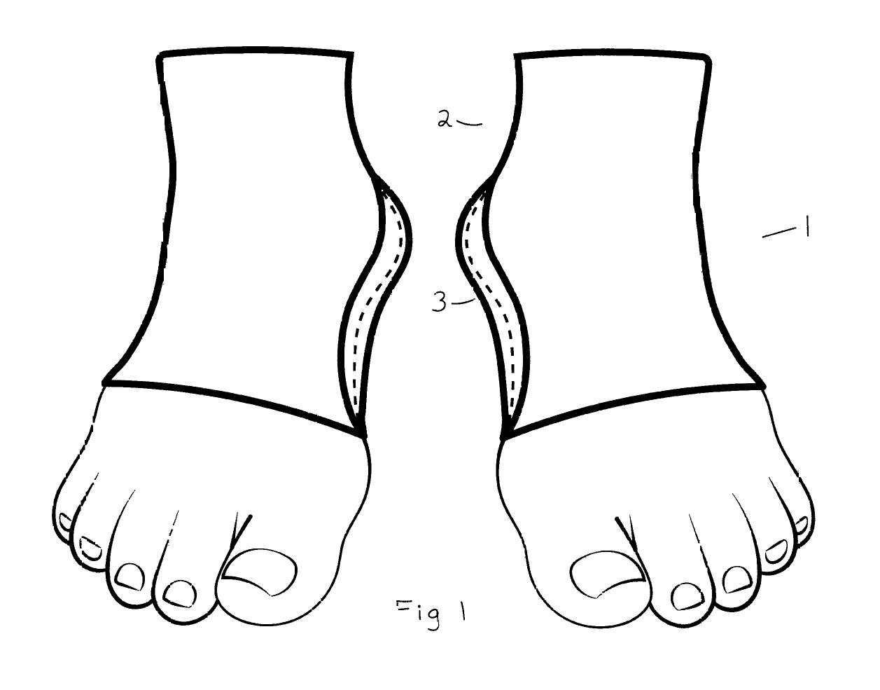

FIG. 1 shows the device 1 comprising an orthotic support structure 3 attached to a brace layer 2. The orthotic support structure 3 is attached to the brace layer 2 by an attaching means such as an adhesive, sewing, or other constructed means. The attaching means is along the entire medial portion length of the orthotic support structure continuously along that length, from the farthest posterior portion of the heel to its farthest anterior portion near the metatarsal heads and along the lateral aspect of the foot, providing a continuous medial and lateral attachment of the orthotic support structure to the brace layer, from it's farthest posterior portion of the heel to its farthest anterior portion near the metatarsal heads.

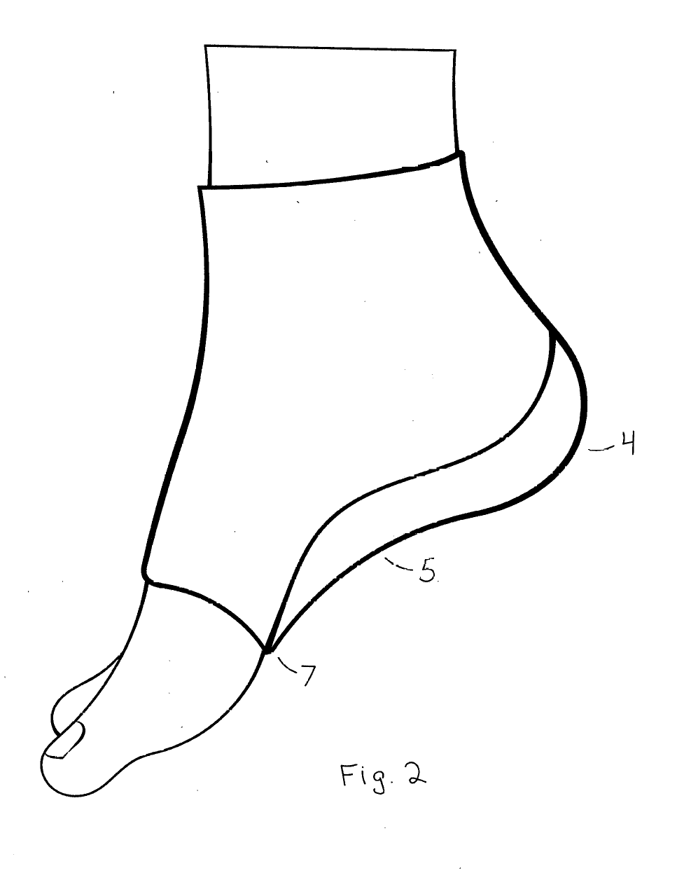

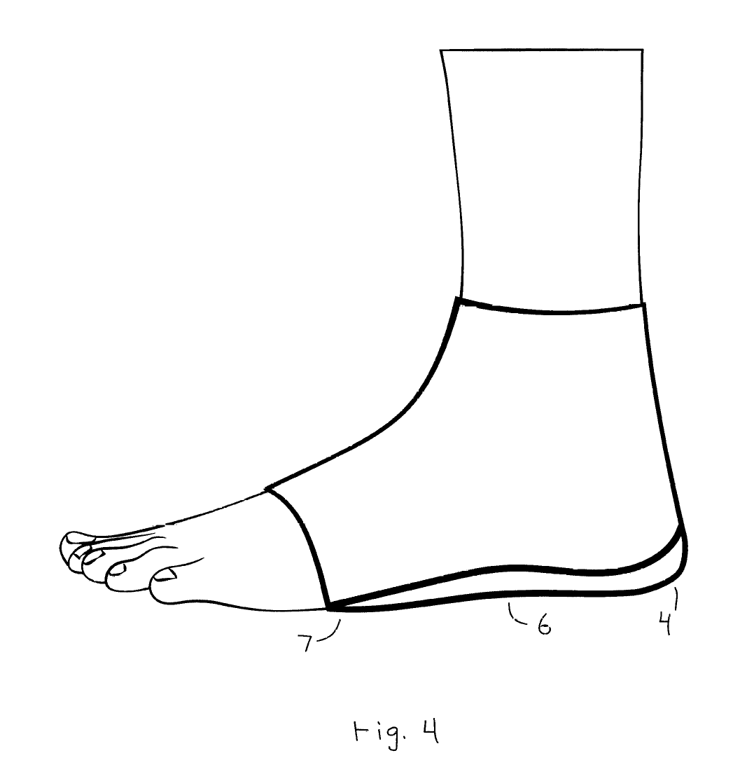

As shown in FIG. 2, the orthotic support structure 3 comprises a tapered heal cup 4, a medial longitudinal arch 5, a tapered end 7, and, as shown in FIG. 4, a lateral longitudinal arch 6.

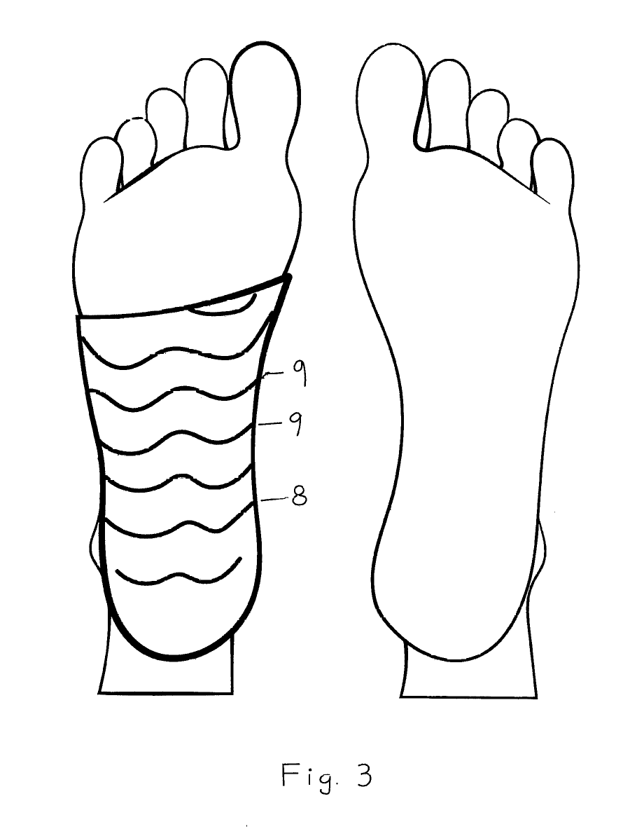

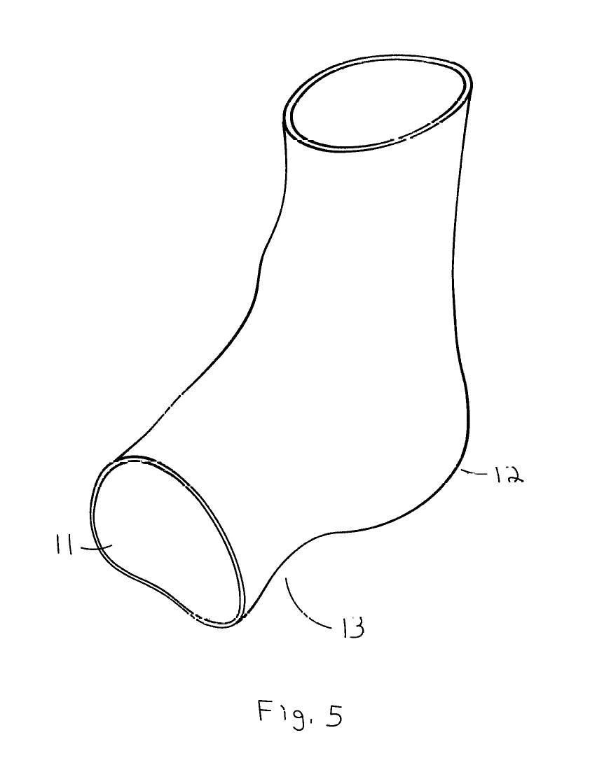

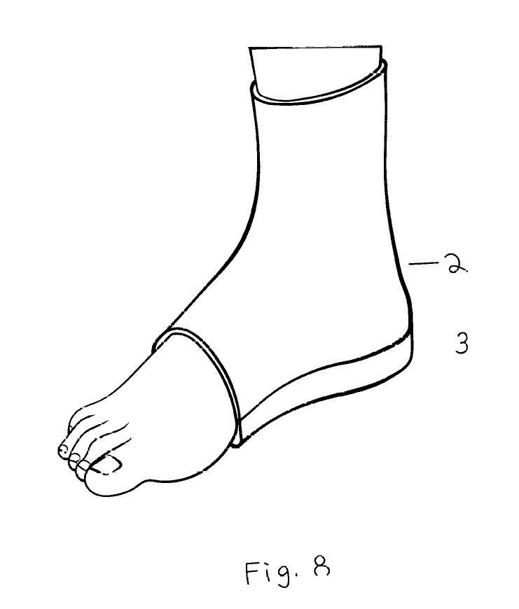

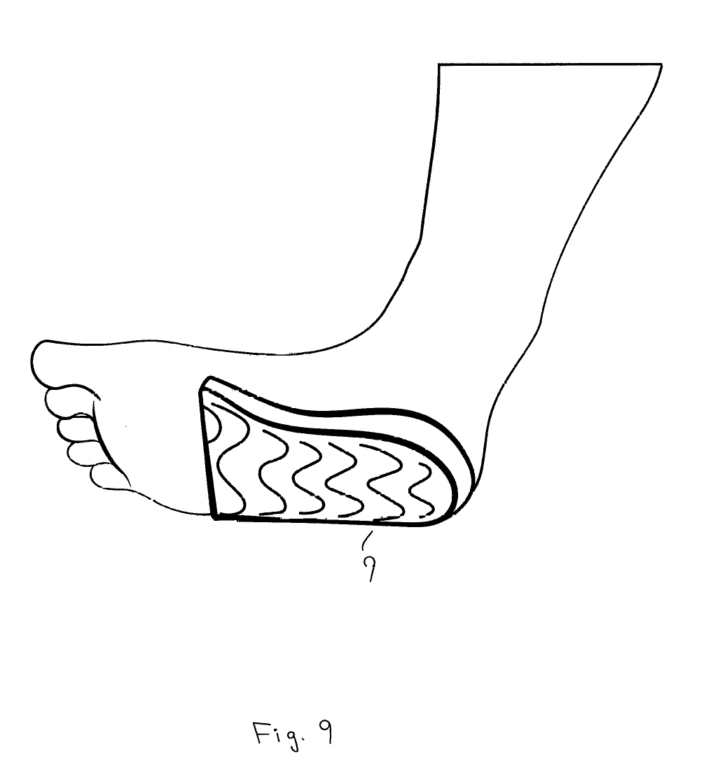

FIG. 3 shows the bottom 8 of the orthotic support structure 3 with anti-slip gripping elements 9. As described earlier, the device has variability in that it can have the brace layer 2 extend completely around the orthotic support structure 3, as shown in FIG. 5, or the orthotic support structure 3 be superficial to the brace layer 2, and provide the previously mentioned gripping properties, as shown in FIGS. 8 and 9. In the event that the brace layer 2 continues around the bottom of the orthotic support structure 3, there may also be gripping elements 9 which are adhered onto the bottom of the brace layer and allow for proper gripping on slick surfaces.

The entire device is meant to be very easily placed on and taken off, and is relatively light in weight, and does not restrict range of motion at the talocrural joint, or the majority of joints at the foot.

As shown in FIG. 5, to place the device on the foot, the wearer places the toes and foot through a top opening 10 of the device. The foot is slid through the device until the forefoot is through the toe opening 11. The wearer's heel at this point should be snug against the heel portion 12 of the device. The brace layer 2 compresses against the majority of the wearer's foot and ankle and holds the orthotic support structure 3 in place to where it is not moveable within the device or in relationship to the wearer's foot. The medial longitudinal arch 5 of the orthotic support structure 3 is flush against the medial longitudinal arch of the wearer's foot. The lateral longitudinal arch 6 of the orthotic support structure 3 lies along the lateral longitudinal arch of the wearer's foot. Upon proper donning of the device, the tapered end 7 of the orthotic support structure 3 is positioned to lie on or just behind the metatarsal heads of the feet. Similarly, the toe opening 11 is a circumferential ending to the device, and lies near the metatarsal heads once properly worn. Upon proper donning of the device, the top opening 10 is positioned to lie at the level or just above the medial and lateral malleoli of the tibia and fibula, respectively. Also shown in FIG. 5 is a brace layer bottom, to which gripping strips or other friction creating means may be attached to prevent the wearer from slipping on slippery surfaces while walking.

This device is meant to contain a custom or generic foot orthotic. Possible customization is attainable by foot scanning devices or measurement deemed accurate to create an orthotic that is specific for the wearer. Therefore, this device is capable of becoming a customizable design and individualized to specific feet.

I claim:

1. An orthotic arch support device designed to be worn on a wearer's foot, comprising an orthotic support structure attached to a brace layer.

2. The device of claim 1 wherein the brace layer comprises a thin, flexible material.

3. The device of claim 1 wherein the orthotic support structure is adhered to the brace layer.

4. The device of claim 1 wherein the orthotic support structure is custom formed to the wearer's foot.

5. The device of claim 1 wherein the orthotic support structure is a generic orthotic.

6. The device of claim 1 wherein the orthotic support structure is comprised of a cushioned type material designed to absorb force from the foot during walking.

7. The device of claim 1 wherein the brace layer is easily placed on by the customer, and likewise easily taken off.

8. The device of claim 1 wherein the orthotic support structure comprises a heel cup, a medial longitudinal arch, a lateral longitudinal arch, a tapered end, and a bottom that conforms to a sole of the wearer's foot.

9. The device of claim 1 wherein the orthotic support structure tapered end extends from mid-foot and covers the medial and lateral longitudinal arches and heel of the foot, the heel cup conforming to the heel of the wearer's foot, without covering the wearer's metatarsal heads or toes of the foot.

10. The device of claim 1 wherein the bottom of the brace is layered or composed of a material with an adhesive or gripping property to allow the device to maintain traction on slippery or low friction surfaces.

11. The device of claim 1 wherein the orthotic support structure extends from above medial and lateral malleoli of a wearer's foot up to the metatarsal heads of the foot.

12. The device of claim 1 wherein the orthotic support structure is incorporated into the manufacturing of the brace layer such that the brace layer surrounds the orthotic support structure and the brace layer further comprises a brace layer bottom.

13. The device of claim 12 wherein gripping strips or other non-friction means are attached to the brace layer bottom.
